(19) United States
(12) Patent Application Publication
(10) Pub. No.: US 2025/0017846 A1
(43) Pub. Date: Jan. 16, 2025

(54) TOPICAL ANALGESICS

(71) Applicant: THE REGENTS OF THE UNIVERSITY OF CALIFORNIA, Oakland, CA (US)

(72) Inventor: Jon Levine, San Francisco, CA (US)

(21) Appl. No.: 18/769,141

(22) Filed: Jul. 10, 2024

Related U.S. Application Data
(60) Provisional application No. 63/512,891, filed on Jul. 10, 2023.

(51) Int. Cl. A61K 9/00; A61K 45/00; A61K 47/20; A61K 47/22; A61K 47/42; A61P 29/00

(52) U.S. Cl. CPC A61K 9/0014; A61K 45/00; A61K 47/20; A61K 47/22; A61K 47/42; A61P 29/00

(57) ABSTRACT

The disclosure is generally related to therapeutic compositions and methods for use in the treatment of somatosensory conditions, including pain and pruritis. In some aspects, the compositions comprise (i) a nonsteroidal anti-inflammatory drug (NSAID), an opioid, or a combination thereof, and (ii) one or more transdermal delivery agents. In some aspects, the compositions comprise (i) an antihistamine, a kappa opioid receptor agonist, an anti-pruritogen, or a combination thereof, and (ii) one or more transdermal delivery agents.

### CROSS-REFERENCE TO RELATED APPLICATIONS

This application claims the priority benefit under 35 U.S.C. § 119 (e) of U.S. Provisional Patent Application No. 63/512,891, filed Jul. 10, 2023, which is incorporated herein by reference in its entirety.

### STATEMENT OF GOVERNMENT INTEREST

This invention was made with government support under R01 CA250017, and R01 AR075334 awarded by the National Institutes of Health. The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “59060A_SeqListing.xml”, which was created on Jul. 9, 2024 and is 10,685 bytes in size. The subject matter of the Sequence Listing is incorporated herein in its entirety by reference.

### BACKGROUND

While there are currently several topical analgesics, both over the counter and prescription, their efficacy is currently limited by the amount of drug that can permeate the stratum corneum layer of the skin, which forms a major barrier for the penetration of large and polar drugs to their site of action.

### SUMMARY

Disclosed herein are various compositions and methods for use in the treatment of somatosensory conditions, including but not limited to pain and pruritis. Advantages provided by the compositions of the disclosure include, but are not limited to, provision of a potent, safe, non-addicting class of topical analgesics and/or anti-pruritics having: 1) rapid onset of action, 2) ability to have high concentrations at the site of action, 3) minimal systemic absorption; and 4) lower likelihood of interactions with other drugs that a subject may be taking for diverse medical conditions.

Accordingly, in some aspects the disclosure provides a composition comprising (i) a nonsteroidal anti-inflammatory drug (NSAID), an opioid, or a combination thereof, and (ii) one or more transdermal delivery agents. In some embodiments, the one or more transdermal delivery agents comprises DMSO, protamine, terpenes, or a combination thereof. In further embodiments, the composition comprises 20-80% DMSO by volume. In some embodiments, the one or more transdermal delivery agents comprises protamine. In further embodiments, the one or more transdermal delivery agents comprises one or more terpenes. In still further embodiments, the one or more terpenes comprises one or more ginkolides. In various embodiments, the ratio of the nonsteroidal anti-inflammatory drug (NSAID), an opioid, or a combination thereof to the one or more transdermal delivery agents is about 0.001 μg per 1 μL to about 10 μg per 1 μl. In some embodiments, the ratio of the nonsteroidal anti-inflammatory drug (NSAID), an opioid, or a combination thereof to the one or more transdermal delivery agents is, is about, is less than about, or is greater than about 0.001 μg per 1 μl, 0.005 μg per 1 μl, 0.010 μg per 1 μl, 0.05 μg per 1 μl, 0.10 μg per 1 μl, 0.5 μg per 1 μl, 1 μg per 1 μl, 2 μg per 1 μl, 5 μg per 1 μl, or 10 μg per 1 μl. In various embodiments, the opioid is oxycodone, oxymorphone, hydrocodone, hydromorphone, fentanyl, sufentanil, alfentanil, remifentanil, morphine, codeine, methadone, tramadol, buprenorphine, meperidine, heroin, tapentadol, oliceridine, nalmefene, naloxone, naltrexone, any compound that targets mu-, kappa-or delta-opioid receptors, or a combination thereof. In some embodiments, the NSAID is actron, aleve, anaprox, ansaid, aspirin acetylsalicylic acid, brufen, butazolidin, cataflam, ceeoxx, celebrex, celecoxib, ceoxx, choline magnesium trisalicylate, clinoril, clotam, daypro, dayrun, dexdetoprofen, diclofenac, diflusinal, disalcid, dolobid, duraprox, dynastat, etodolac, etoricoxib, feldene, fenoprofen, firocoxib, flurbiprofen, flurwood, froben, ibuprofen, indomethacin, keral, ketoflam, ketrolac, licofelone, lodine, lornoxicam, loxoprofen, loxonin, loxomac, lumiracoxib, meclomen, meclofenamic acid, meclofenemate, medipren, mefenamic acid, melox, meloxicam, mesulid, midol, mobic, mobiflex, mono-gesic, motrin, movalis, nabumetone, naprelan, naprosyn, naproxen, nimalox, nimesulide, nuprin, nurofen, orudis, oruvail, oxaporozin, oxeno, parecoxib, phenylbutazone, piroxicam, ponstel, previcox, prexige, rapid, recoxa, relafen, rofecoxib, salflex, salicylate, salsalate, salsitab, sprix, sulide, sulindac, tenoxicam, tolectin, tolfenamic acid, toradol, trilisate disaclid, tufnil, urbifen, valdecoxib, vioxx, voltaren xefo, any compound that targets COX-1 or COX-2, or a combination thereof. In further aspects, the disclosure provides methods of treating a pain condition in a subject in need thereof, the method comprising topically administering to the subject a composition as disclosed herein. Thus, in various aspects, the disclosure provides methods of treating a pain condition in a subject in need thereof, the method comprising topically administering to the subject a composition comprising (i) a nonsteroidal anti-inflammatory drug (NSAID), an opioid, or a combination thereof, and (ii) one or more transdermal delivery agents. In some embodiments, the pain condition is neuropathic pain, inflammatory pain, chemotherapy-induced painful peripheral neuropathy (CIPN), stress-induced pain, or a combination thereof.

In further aspects, the disclosure provides a composition comprising (i) an antihistamine, a kappa opioid receptor agonist, an anti-pruritogen, or a combination thereof, and (ii) one or more transdermal delivery agents. In some embodiments, the one or more transdermal delivery agents comprises DMSO, protamine, terpenes, or a combination thereof. In further embodiments, the composition comprises 20-90% DMSO by volume. In some embodiments, the one or more transdermal delivery agents comprises protamine. In further embodiments, the one or more transdermal delivery agents comprises one or more terpenes. In still further embodiments, the one or more terpenes comprises one or more ginkolides. In various embodiments, the ratio of the antihistamine, the kappa opioid receptor agonist, the anti-pruritogen, or combination thereof to the one or more transdermal delivery agents is about 0.001 μg per 1 μL to about 10 μg per 1 μl. In some embodiments, the ratio of the antihistamine, the kappa opioid receptor agonist, the anti-pruritogen, or combination thereof to the one or more transdermal delivery agents is, is about, is less than about, or is greater than about 0.001 μg per 1 μl, 0.005 μg per 1 μl, 0.010 μg per 1 μl, 0.05 μg per 1 μl, 0.10 μg per 1 μl, 0.5 μg per 1 μl, 1 μg per 1 μl, 2 μg per 1 μl, 5 μg per 1 μl, or 10 μg per 1 μl. In various embodiments, the antihistamine is chlorpheniramine, brompheniramine, dexchlorpheniramine, tripolidine, clemastine, diphenhydramine, promethazine, piperazines, piperidines, astemizole, loratadine and terfenadine. Further examples of antihistamines contemplated by the disclosure include, but are not limited to, Brompheniramine (Dimetane), Cetirizine (Zyrtec), Chlorpheniramine (Chlor-Trimeton), Clemastine (Tavist), Diphenhydramine (Benadryl), Fexofenadine (Allegra), or Loratadine (Alavert, Claritin)); adrenaline, or a combination thereof. In various embodiments, the kappa opioid receptor agonist is JT09, nalfurafine, CR665, or CR845, nalbuphine, butorphanol, pentazocine, asimadoline, enadoline, fedotozine, spiradoline, nalfurafine, noribogaine, niravoline, difelikefalin, apadoline, salvinorin A, levorphanol, levallorphan, phenazocine, eptazocine, CR665 (FE-200665, JNJ-38488502), or a combination thereof. In further embodiments, the anti-pruritogen is hydroxyzine, benadryl, diphenhydramine, gabapentin, vistaril, hydrocortisone, doxepin, ondansetron, ammonium lactate, calamine, cyproheptadine, diphenhyramine, amlactin, banophen, cortizone, pramoxine, ala-cort, diphedryl, eurax, lac-hydrin, xylocaine, ala-scalp, allermax, anecream, anusol, aquanil, aquax, bactine, cidaleaze, crotamiton, dermacinrx lidotral, dermacinrx lido v pak, dermarest plus anti-itch, dermtex hc, dicopanol, diphen, dytuss, eha lotion, gen7t lotion, gynecort, itch-x lotion, itch relief, lidamantle, lidogel, lidozion, locoid, lydexa, nalbuphine, difelikefalin, asamadoline, nalfurafine, butorphanol, pentazocine, nucort, pandel, proctozone, prudoxin, regencare, sarna sensitive, scot-tussin, texacorttwilite, u-cort, valu-dryl, vanamine, pdzonalon, ala-quin, ana-lex, anacaine, anamantle, benzocaine, chiggerex, geri-hydrolac, gold bond, kerasal al, lanacane, menthol, pramox, prax, radiaura, or a combination thereof. In further aspects, the disclosure provides methods of treating pruritus in a subject in need thereof, the method comprising topically administering to the subject a composition as disclosed herein. Thus, in various aspects, the disclosure provides methods of treating pruritus in a subject in need thereof, the method comprising topically administering to the subject a composition comprising (i) an antihistamine, a kappa opioid receptor agonist, an anti-pruritogen, or a combination thereof, and (ii) one or more transdermal delivery agents.

### BRIEF DESCRIPTION OF THE DRAWINGS

FIG. 1 shows that topical morphine induced anti-hyperalgesia in rats with chemotherapy-induced painful peripheral neuropathy (CIPN). A. Oxaliplatin decreased mechanical nociceptive threshold (i.e., produced hyperalgesia), and topical administration of morphine in protamine vehicle attenuated oxaliplatin-induced hyperalgesia in female rats. Two-way repeated measures ANOVA, F_{(6.18)}=26.85, P<0.0001. Bonferroni's multiple comparison post-hoc test. n=3 per group. B. Oxaliplatin decreased mechanical nociceptive threshold (i.e., produced hyperalgesia), and topical administration of morphine attenuated oxaliplatin-induced hyperalgesia in male rats. Two-way repeated measures ANOVA, F_{(6.36)}=15.21, P<0.0001. Bonferroni's multiple comparison post-hoc test. n=6 per group.

FIG. 2 shows that topical morphine induced anti-PGE2 hyperalgesia.

FIG. 3 shows that topical indomethacin induced anti-hyperalgesia.

### DETAILED DESCRIPTION

In various aspects, the present disclosure provides compositions and methods for treating somatosensory conditions, including but not limited to pain and pruritis. In some aspects, the methods comprise administering an agent as described herein to a subject suffering from a condition such as pain and/or pruritis. In some aspects, the methods of the disclosure combine diverse drugs known to act in peripheral tissues to produce analgesia, with a transdermal drug delivery enhancer, to produce a class of highly effective topical analgesics that do not affect baseline nociceptive threshold (i.e., are not local anesthetics). In various aspects, compositions of the disclosure comprise diverse analgesic and/or anti-pruritic drugs that have therapeutic effects by their action in peripheral tissues (i.e., outside the central nervous system).

The disclosure contemplates use of one or more agents as described herein. In any of the aspects or embodiments of the disclosure, the one or more agents is provided in a therapeutically effective amount. A therapeutically effective amount of an agent is an amount that has one or more selected biological or therapeutic effects. In some embodiments, the biological or therapeutic effect is an anti-hyperalgesia effect. In some embodiments, the biological or therapeutic effect is an anti-pruritic effect.

A “subject” is a vertebrate organism. The subject can be a non-human mammal (e.g., a mouse, a rat, or a non-human primate), or the subject can be a human subject. The subject may be any non-human animal, such as a test animal, pet, livestock, or veterinary subject.

A “therapeutically effective amount” as used herein refers to an amount sufficient to have any measurable therapeutic effect. For example, in some embodiments, the topical administration comprises the administration of about, at least about, or less than about 0.001 μg to about 1.0 gram (g), 0.005 μg to about 1.0 g, 0.1 μg to 1.0 g, about 0.2 μg to 1.0 g, about 0.3 μg to 1.0 g, about 0.4 μg to 1.0 g, about 0.5 μg to 1.0 g, about 0.6 μg to 1.0 g, about 0.7 μg to 1.0 g, about 0.8 μg to 1.0 g, about 0.9 μg to 1.0 g, 1.0 μg to 1.0 gram, for example, about, at least about, or less than about, 0.1 μg, 0.2 μg, 0.3 μg, 0.4 μg, 0.5 μg, 0.6 μg, 0.7 μg, 0.8 μg, 0.9 μg, 1 μg, 2 μg, 3 μg, 4 μg, 5 μg, 6 μg, 7 μg, 8 μg, 9 μg, 10 μg, 20 μg, 30 μg, 40 μg, 50 μg, 60 μg, 70 μg, 80 μg, 90 μg, 100 μg, 120 μg, 150 μg, 200 μg, 220 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1 mg, 5 mg, 10 mg, 15 mg, 20 mg, 30 mg 40 mg, 50 mg, 60 mg, 70 mg, 80 mg, 90 mg, 100 mg, 250 mg, 500 mg, 750 mg, or 1 g of the agent per dose, for example 0.001 μg-100 μg of the agent per square centimeter of skin surface. The foregoing dosages can refer to the dosage of a single agent when a composition comprises a single agent, or the foregoing dosages can refer to the dosage of each of two or more agents when a composition comprises more than one agent, or the foregoing dosages can refer to the total dosage of two or more agents when a composition comprises more than one agent. Depending on dosage form, condition, and other factors, administrations may be, for example, multiple times per day, once per day, every other day, once per week, or otherwise as needed.

As used in this specification and the appended claims, the singular forms “a,” “an,” and “the” include plural reference unless the context clearly dictates otherwise.

All language such as “from,” “to,” “up to,” “at least,” “greater than,” “less than,” and the like include the number recited and refer to ranges which can subsequently be broken down into sub-ranges.

A range includes each individual member. Thus, for example, a group having 1-3 members refers to groups having 1, 2, or 3 members. Similarly, a group having 6 members refers to groups having 1, 2, 3, 4, or 6 members, and so forth.

“About” and “approximately” shall generally mean an acceptable degree of error for the quantity measured given the nature or precision of the measurements. Exemplary degrees of error are within 20-25 percent (%), for example, within 20 percent, 10 percent, 5 percent, 4 percent, 3 percent, 2 percent, or 1 percent of the stated value or range of values.

In any of the aspects or embodiments of the disclosure, “administration” of an agent as described herein (e.g., a composition, a NSAID, an opioid, an anti-pruritogen) is topical, for example, administered to the skin by exposure thereto. Topical administration is advantageously non-invasive and is amenable to convenient self-administration. Exemplary administrations include the application, for example, by hand, of lotions, ointments, salves, creams or other like dosage forms or topical application by a patch (e.g., an adhesive patch) or dressing coated or saturated with a therapeutic agent of the disclosure. Administration will generally be localized to the area where an adverse condition (e.g., pain condition, pruritis condition, inflammatory condition) is being experienced, for example, to the hands, face, feet, limbs or other afflicted area or into body cavities, such as joints, abdomen and thorax. In various embodiments, the administration may be by subdermal injection, injection by microneedles, sonic-assisted delivery, and other methods of delivering a therapeutic agent to, for example, pruriceptors wherein pruritus is being perceived. In some embodiments the administration is to an internal compartment of the body, for example, by injection, infusion, or other administration, for example, to body cavities: joints, the abdominal cavity, and/or into the gastrointestinal tract.

As used herein, “treating” and “treatment” refers to any reduction in the severity and/or onset of symptoms associated with a condition, disease, or disorder (e.g., pain, pruritis). Accordingly, “treating” and “treatment” includes therapeutic and prophylactic measures. One of ordinary skill in the art will appreciate that any degree of protection from, or amelioration of, the condition (e.g., pain, pruritis) is beneficial to a subject, such as a human patient. The quality of life of a patient is improved by reducing to any degree the severity of symptoms in a subject and/or delaying the appearance of symptoms.

### COMPOSITIONS

The compositions of the disclosure may comprise any formulation capable of effective topical application. Exemplary dosage forms include creams, salves, ointments, drug delivery patches, and other dosage forms known in the art for topical delivery of agents. Compositions of the disclosure generally comprise one or more agents (e.g., a nonsteroidal anti-inflammatory drug (NSAID), an opioid, an antihistamine, a kappa opioid receptor agonist, an anti-pruritogen, or a combination thereof) and one or more transdermal delivery agents (e.g., DMSO, protamine, terpenes, or a combination thereof).

It will be understood that the topical compositions of the disclosure, in addition to comprising one or more agents and one or more transdermal delivery agents, may further include any other constituents, for example, carriers, timed-release compositions, fillers, excipients, coloring agents, scents, and other compositions of matter. The topical compositions of the disclosure may further comprise combination products, for example, including additional active agents, such as topical anesthetics, anti-inflammatory agents, moisturizers, sunscreen, and other agents that augment or supplement the therapeutic properties of the compositions.

“Agents” that are useful in the compositions of the disclosure include, but are not limited to, a nonsteroidal anti-inflammatory drug (NSAID), an opioid, an antihistamine, a kappa opioid receptor agonist, an anti-pruritogen, or a combination thereof.

In some aspects, the disclosure provides a composition comprising (i) a nonsteroidal anti-inflammatory drug (NSAID), an opioid, or a combination thereof, and (ii) one or more transdermal delivery agents.

Nonsteroidal anti-inflammatory drug (NSAID). NSAIDs contemplated by the disclosure include, but are not limited to, actron, aleve, anaprox, ansaid, aspirin acetylsalicylic acid, brufen, butazolidin, cataflam, ceeoxx, celebrex, celecoxib, ceoxx, choline magnesium trisalicylate, clinoril, clotam, daypro, dayrun, dexdetoprofen, diclofenac, diflusinal, disalcid, dolobid, duraprox, dynastat, etodolac, etoricoxib, feldene, fenoprofen, firocoxib, flurbiprofen, flurwood, froben, ibuprofen, indomethacin, keral, ketoflam, ketrolac, licofelone, lodine, lornoxicam, loxoprofen, loxonin, loxomac, lumiracoxib, meclomen, meclofenamic acid, meclofenemate, medipren, mefenamic acid, melox, meloxicam, mesulid, midol, mobic, mobiflex, mono-gesic, motrin, movalis, nabumetone, naprelan, naprosyn, naproxen, nimalox, nimesulide, nuprin, nurofen, orudis, oruvail, oxaporozin, oxeno, parecoxib, phenylbutazone, piroxicam, ponstel, previcox, prexige, rapid, recoxa, relafen, rofecoxib, salflex, salicylate, salsalate, salsitab, sprix, sulide, sulindac, tenoxicam, tolectin, tolfenamic acid, toradol, trilisate disaclid, tufnil, urbifen, valdecoxib, vioxx, voltaren xefo, any compound that targets COX-1 or COX-2, or a combination thereof.

Opioid. As used herein, the term “opioid” refers to all agonists and antagonists of opioid receptors, such as mu (μ), kappa (κ), and delta (δ) opioid receptors and subtypes thereof. Opioids contemplated by the disclosure include, but are not limited to, oxycodone, oxymorphone, hydrocodone, hydromorphone, fentanyl, sufentanil, alfentanil, remifentanil, morphine, codeine, methadone, tramadol, buprenorphine, meperidine, heroin, tapentadol, oliceridine, nalmefene, naloxone, naltrexone, any compound that targets mu-, kappa-or delta-opioid receptors, or a combination thereof.

In some aspects, the disclosure provides compositions comprising (i) an antihistamine, a kappa opioid receptor agonist, an anti-pruritogen, or a combination thereof, and (ii) one or more transdermal delivery agents.

Antihistamine. Antihistamines contemplated for use according to the disclosure include, but are not limited to, any of various compounds that counteract histamine in the body. Non-limiting examples of antihistamines usable in context of the described invention include chlorpheniramine, brompheniramine, dexchlorpheniramine, tripolidine, clemastine, diphenhydramine, promethazine, piperazines, piperidines, astemizole, loratadine and terfenadine. Further examples of antihistamines contemplated by the disclosure include, but are not limited to, Brompheniramine (Dimetane), Cetirizine (Zyrtec), Chlorpheniramine (Chlor-Trimeton), Clemastine (Tavist), Diphenhydramine (Benadryl), Fexofenadine (Allegra), or Loratadine (Alavert, Claritin)); adrenaline, or a combination thereof.

Kappa opioid receptor agonist (KOA). The disclosure contemplates that any pharmacologically acceptable kappa opioid agonist will function in the compositions and methods of the disclosure. Such kappa opioid receptor agonists bind to and stimulate kappa opioid receptors. In various embodiments, the KOA comprises an organic small molecule. Non-limiting examples of organic small molecules comprise JT09, nalfurafine, CR665, CR845, nalbuphine, butorphanol, pentazocine, asimadoline, enadoline, fedotozine, spiradoline, nalfurafine, noribogaine, niravoline, difelikefalin, apadoline, salvinorin A, levorphanol, levallorphan, phenazocine, eptazocine. and CR665 (FE-200665, JNJ-38488502).

Anti-pruritogen. The term “anti-pruritogen” is used interchangeably herein with the term “anti-itch” and refers to any compound that reduces, ameliorates, or eliminates itch in a subject. In various aspects, the disclosure contemplates that any compound that targets the following receptors may be used in the compositions and methods of the disclosure: Histamine (H1R, H4R), MrgprC11, MrgprA3, MrgprC11, PAR2, MrgprD, PAR2, PAR4, 5-HT1, 5-HT2, ETA, NK1, IL-31RA, OSMR, TLR3, TGR5, or a combination thereof. Specific anti-pruritogens contemplated for use in the compositions and methods of the disclosure include, but are not limited to, hydroxyzine, benadryl, diphenhydramine, gabapentin, vistaril, hydrocortisone, doxepin, ondansetron, ammonium lactate, calamine, cyproheptadine, diphenhyramine, amlactin, banophen, cortizone, pramoxine, ala-cort, diphedryl, eurax, lac-hydrin, xylocaine, ala-scalp, allermax, anecream, anusol, aquanil, aquax, bactine, cidaleaze, crotamiton, dermacinrx lidotral, dermacinrx lido v pak, dermarest plus anti-itch, dermtex hc, dicopanol, diphen, dytuss, eha lotion, gen7t lotion, gynecort, itch-x lotion, itch relief, lidamantle, lidogel, lidozion, locoid, lydexa, nalbuphine, difelikefalin, asamadoline, nalfurafine, butorphanol, pentazocine, nucort, pandel, proctozone, prudoxin, regencare, sarna sensitive, scot-tussin, texacorttwilite, u-cort, valu-dryl, vanamine, pdzonalon, ala-quin, ana-lex, anacaine, anamantle, benzocaine, chiggerex, geri-hydrolac, gold bond, kerasal al, lanacane, menthol, pramox, prax, radiaura, or a combination thereof.

### Transdermal Delivery Agents

In various aspects and embodiments, compositions and methods of the disclosure comprise use of one or more transdermal delivery agents. The transdermal delivery agent, as used herein, comprises a material that facilitates transit of an agent of the disclosure across the epidermis to target nociceptors and/or pruriceptors of the dermis. Any number of surfactants, solvents, skin penetrating agents, and other compositions known in the art for transdermal delivery may be utilized.

In some embodiments, the transdermal delivery agent will synergize or otherwise improve the efficacy of the delivered agent (e.g., a nonsteroidal anti-inflammatory drug (NSAID), an opioid, an antihistamine, a kappa opioid receptor agonist, an anti-pruritogen, or a combination thereof). In some embodiments, the transdermal delivery agent synergizes or otherwise improves the efficacy of the delivered agent by enhancing and/or prolonging the therapeutic effects of the agent. In various embodiments, the transdermal delivery agent comprises DMSO, protamine, terpenes, or a combination thereof.

DMSO. In some embodiments, the transdermal delivery agent is dimethyl sulfoxide (DMSO). DMSO is known in the art as an effective agent for facilitating transdermal delivery of various agents. In some embodiments, the disclosure provides a composition comprising a solution of an agent in a solvent; and DMSO.

Any transdermal delivery agent known in the art may be used in the topical compositions of the disclosure. Exemplary transdermal delivery agents include but are not limited to:
Protamine and variants thereof;
Terpenes, for example: thymol, tetra-hydrogeraniol, pulegone; nerolidol; menthol; linalool; farnesol; carvone; camphor; and anethole;
Pyrrolidones, for example: N-methylpyrrolidone; Poly [acrylonitrile-co-(N-vinyl pyrrolidone)]; and 2-pyrrolidone;
Liposomes or other nanoparticles, such as nanoparticles comprising: chitosan; polyalkylcyanoacylates; poly-lactic acid; poly-e-caprolactone; poly-glycolic acid; and poly-lactic-co-glycolic acid (PLGA);
Skin penetrating peptides, such as Peptide R7 (Polyarginine-7); YARA (YARAAARQARA (SEQ ID NO: 1)); WLR (WLRRIKAWLRRIKAWLRRIKA (SEQ ID NO: 2)); TAT (YGRKKRRQRRR (SEQ ID NO: 3)); Penetratin (RQIKIYFQNRRMKWKK (SEQ ID NO: 4)); VP22 (DAATATRGRSAASRPTERPRAPARSASRPRRPVE (SEQ ID NO: 5)); Membrane translocating sequence peptide (AAVALLPAVLLALLAP (SEQ ID NO: 6)); Transportan (GWTLNSAGYLLKINLKALAALAKKIL (SEQ ID NO: 7)); Polyarginine (RRRRRRRRRRR (SEQ ID NO: 8)); Model amphipathic peptide (KLALKLALKALKAALKLA (SEQ ID NO: 9)); Flock house virus (RRRRNRTRRNRRRVR (SEQ ID NO: 10)); and Pep-1(hydrophobic/NLS) (KETWWETWWTEWSQPKKKRKV (SEQ ID NO: 11));
other arginine oligomers, including Rothbard et al., 2000, Conjugation of arginine oligomers to cyclosporin A facilitates topical delivery and inhibition of inflammation: Nature Medicine 6:1253-1257;
long-chain fatty acids, such as: oleic acid; lauric acid; myristic acid; and capric acid;
glycols, such as diethylene glycol and tetraethylene glycol;
surfactants, such as: polyoxyethylene-2-oleyl ether; and polyoxy ethylene-2-stearly ether;
essential oils, such as oils of: eucalyptus; Chenopodium; and ylang-ylang; and
other transdermal penetration agents, such as: 4-decyloxazolidin-2-one; azone; urea, and nicotinamide.

Protamine. In some embodiments, the transdermal delivery agent comprises protamine. Protamines are small polycationic proteins. Protamines are produced in the spermatids of many species and aid in the compact packaging of DNA. The disclosure contemplates the use of any protamines of any species or artificial protamine, or protamine derivative, including, for example, monoprotamines, diprotamines, triprotamines, fish protamines such as Salmine or Clupeine, protamine in free base form, protamine salts such as protamine sulfate and protamine chloride, low-molecular-weight protamine forms, polyethylene glycol (PEG)-conjugated protamines, and Mannosylated protamine sulfate.

In the compositions and methods of the disclosure, protamine may be combined with one or more agents by any process. By way of example, an agent of the disclosure may be combined with protamine as follows: A stock solution of an agent is made by dissolving the agent in distilled water. Protamine is dissolved in distilled water (dH₂O) to a concentration of about 5 μg/μl, then the stock solution of the agent is combined with the protamine solution at a desired final concentration (for example and without limitation, 2 μg/μl in a volume of 30 μl, administered topically).

Terpenes, such as Ginkolides. In some embodiments, the transdermal delivery agent is a terpene. In further embodiments, the terpenes are terpenes derived from Ginko spp. In various embodiments, the terpenes comprise one or more of:

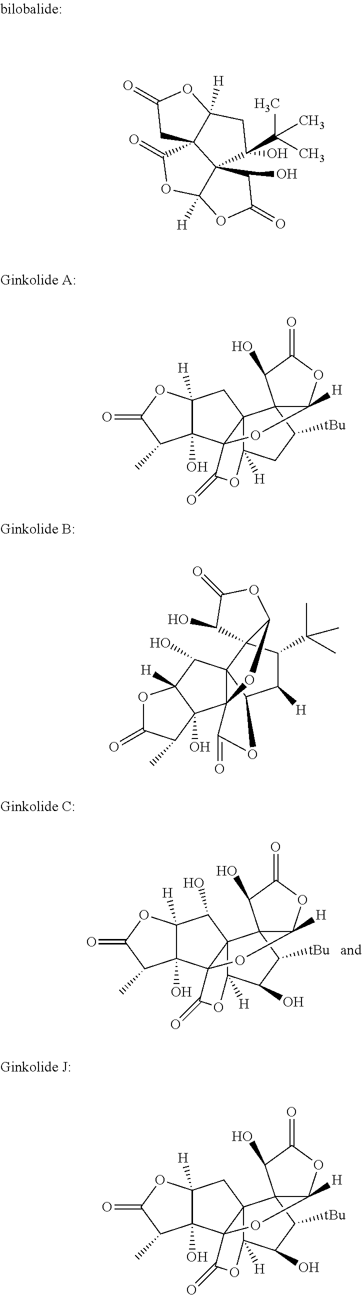

Additional terpenes that may be utilized as the transdermal delivery agent are known in the art, for example, as described in Chen et al. (2016) Natural Terpenes as Penetration Enhancers for Transdermal Drug Delivery. Molecules 21: 1709.

In the formulations and methods of the disclosure, terpenes, e.g., ginkolides, may be combined with an agent by any process. By way of example, terpenes comprising a mixture of ginkolides is first dissolved in dH₂O at a concentration of about 2 μg/μl, then an agent is dissolved in this stock solution of terpene to a desired final concentration (for example, 2 μg/μl in a volume of 30 μl, administered topically).

### Methods

In various aspects, the disclosure provides methods of using the agents and compositions as described herein to treat a subject in need thereof.

Treatment of pain. In some aspects, the disclosure provides methods to treat pain in a subject in need thereof. Thus, in some aspects, the disclosure provides a method of treating pain in a subject in need of treatment for a pain condition comprising administering an agent of the disclosure (e.g., a composition as described herein comprising an agent) to the subject.

In various embodiments, the pain condition is any condition wherein an agent as described herein will alleviate the condition. Alleviation, as used herein, encompasses any reduction in pain, hyperalgesia or allodynia, reduction in the perception of pain, inhibition of processes which underlie the condition; or any other therapeutic effect with regard to the condition. The pain to be alleviated may be of any type, including but not limited to neuropathic, nociceptive, inflammatory, stress-related, psychogenic, or functional pain.

In some embodiments, the pain condition is neuropathic pain. Exemplary forms of neuropathic pain include, for example: chemotherapy-induced neuropathic pain; peripheral neuropathy; focal neuropathy, autonomic neuropathy; polyneuropathy, proximal neuropathy, diabetic neuropathy, trigeminal neuralgia; small-fiber neuropathy, and compression mononeuropathy. Other forms of neuropathy include CRPS-1, CRPS-2, drug-induced, infectious, alcohol-induced, idiopathic, immune-mediated, and traumatic neuropathy.

In some embodiments, the pain condition is inflammatory pain, i.e., pain associated with inflammation or reparatory processes in response to injury, for example, pain associated with peripheral sensitization.

In further embodiments, the pain condition is a condition resulting from trauma, injury, surgery, disease, allergy, infection, or other pain-causing factors. Exemplary conditions include stress-related pain: fibromyalgia syndrome, pain associated with irritable bowel syndrome, pain associated with post-traumatic stress-disorder (PTSD), and dry eye syndrome.

Treatment of Pruritus. In some aspects, the disclosure provides methods to treat pruritis in a subject in need thereof. Thus, in some aspects, the disclosure provides a method of treating pruritis in a subject in need of treatment for a pruritis condition comprising administering an agent of the disclosure (e.g., a composition as described herein comprising an agent) to the subject.

The pruritus condition may be any condition encompassing itch or like sensations wherein an agent of the disclosure will alleviate the condition. Alleviation, as used herein, encompasses any reduction in itch, reduction in the perception of itch, inhibition of processes which underlie the condition; or any other therapeutic effect with regard to the condition.

The pruritus may be of any type or by any cause. Exemplary forms of pruritus include: idiopathic generalized pruritus; allergic contact dermatitis; atopic dermatitis; urticaria; pruritus associated with psoriasis; pruritus associated with dry skin; advanced age or senile pruritus; pruritus associated with sunburn; pruritus from insect bite or other biting organism; systemic pruritus; renal or uremic pruritus; cholestatic pruritus; pruritus associated with thyroid conditions; pruritus associated with cancer; pruritis associated with conditions of the eye; brachioradial pruritus; pruritus associated with shingles or peripheral neuropathy; pruritus ani; and psychogenic or somatoform pruritus.

The following examples are given merely to illustrate the present disclosure and not in any way to limit its scope.

### EXAMPLES

### Example 1

In this Example, data and figures are provided that demonstrate the ability of combining an opioid (morphine) and a NSAID (indomethacin) with the transdermal drug delivery enhancer protamine, but not without protamine, to produce highly effective topical analgesia. This example provides support for the use of the combination of agents (e.g., peripherally acting drugs) with transdermal drug enhancers as a novel class of topical therapeutics for use as an analgesic and/or an anti-pruritic therapeutic.

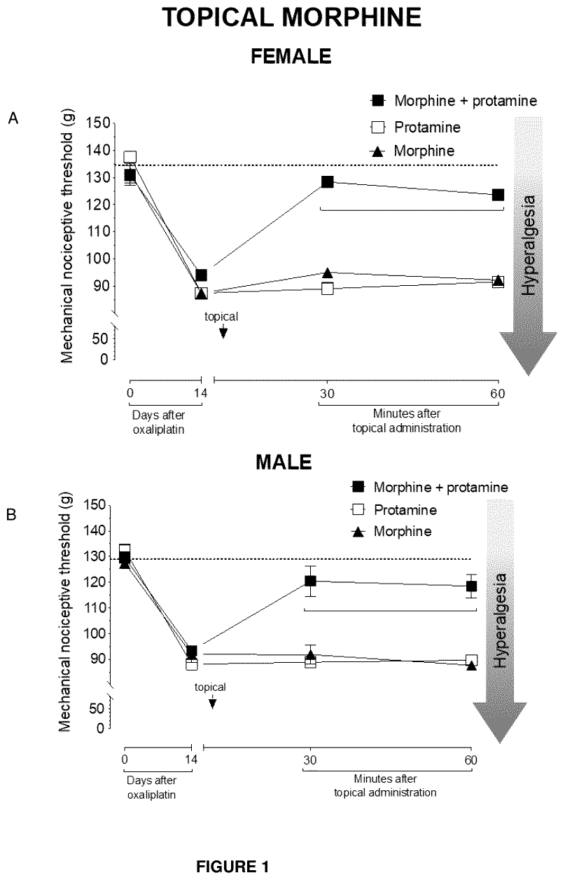

FIG. 1 shows that topical morphine induced anti-hyperalgesia in rats with chemotherapy-induced painful peripheral neuropathy (CIPN). Female rats received oxaliplatin (2 mg/kg, i.v.) on day 0. On day 14, at which time oxaliplatin-induced hyperalgesia was established, morphine combined with protamine or protamine alone (dissolved in distillated water-dH₂0) or morphine (using saline as a vehicle), each in a volume of 30 μL, was administered topically on the dorsum of one hind paw, at the site of nociceptive threshold testing. Protamine was used at a concentration of 2 μg/μl and morphine was used at a concentration of 0.1 μg/μl. Mechanical nociceptive threshold was evaluated before and 14 days after oxaliplatin administration, and again 30 and 60 minutes after topical drug administration. Oxaliplatin decreased mechanical nociceptive threshold (i.e., produced hyperalgesia), and topical administration of morphine in protamine vehicle attenuated oxaliplatin-induced hyperalgesia. Two-way repeated measures ANOVA, F₍6.18)=26.85, P<0.0001. Bonferroni's multiple comparison post-hoc test. n=3 per group. See FIG. 1A.

Male rats received oxaliplatin (2 mg/kg, i.v.) on day 0. On day 14, at which time oxaliplatin-induced hyperalgesia was established, morphine combined with protamine or protamine alone, each in a volume of 30 μL, was administered topically on the dorsum of one hind paw, at the site of nociceptive threshold testing. Protamine was used at a concentration of 2 μg/μl and morphine was used at a concentration of 0.1 μg/μl. Mechanical nociceptive threshold was evaluated before and 14 days after oxaliplatin administration, and again 30 and 60 minutes after topical administration. Oxaliplatin decreased mechanical nociceptive threshold (i.e., produced hyperalgesia), and topical administration of morphine attenuated oxaliplatin-induced hyperalgesia. Two-way repeated measures ANOVA, F₍6.36)=15.21, P<0.0001. Bonferroni's multiple comparison post-hoc test. n=6 per group. See FIG. 1B.

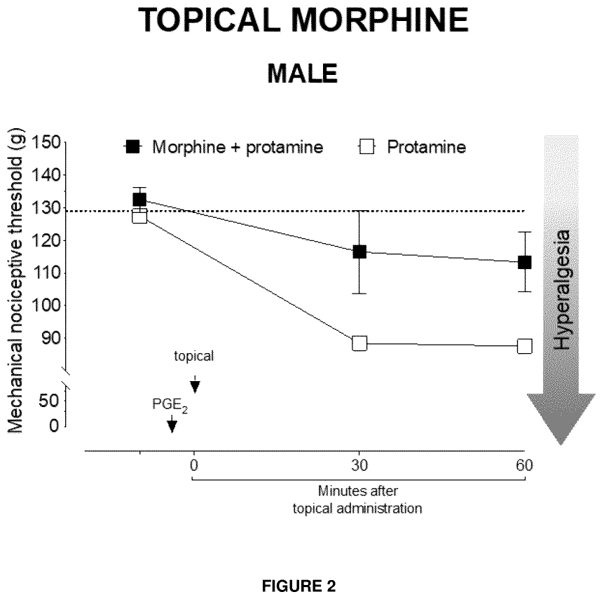

FIG. 2 shows that topical morphine induced anti-PGE₂ hyperalgesia. Male rats received PGE₂ (100 ng/5 μL, intradermally, i.d.), on the dorsum of the hind paw followed, 10 minutes later, by morphine combined with protamine or protamine alone (dissolved in distillated water-dH₂0) or morphine (using saline as a vehicle), each in a volume of 30 μL, was administered topically on the dorsum of one hind paw, at the site of the i.d. PGE₂ injection. Protamine was used at a concentration of 2 μg/μl and morphine was used at a concentration of 0.1 μg/μl. Mechanical nociceptive threshold was evaluated before intradermal PGE₂ and again 30 and 60 min after topical administration. Measurement of mechanical nociceptive threshold showed a significant attenuation of PGE2-hyperalgesia in rats treated with topical morphine. Two-way repeated measures ANOVA, F₍2.8)=4.25, *P=0.0366. Bonferroni's multiple comparison post-hoc test. Data shown as mean±SEM; n=3 paws, per group.

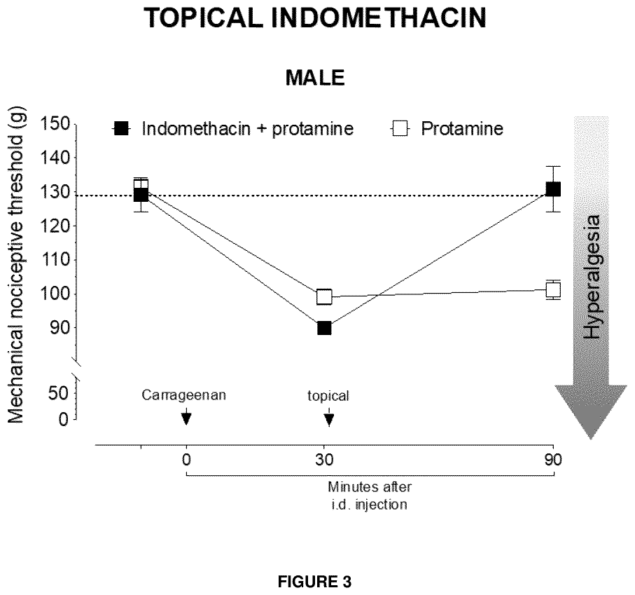

FIG. 3 shows that topical indomethacin induced anti-hyperalgesia. Male rats were treated with carrageenan (0.1% in 1 μg/5 μL) intradermally (i.d.), into the dorsum of the hind paw. Thirty minutes after the carrageenan administration, when hyperalgesia was established, animals received indomethacin combined with protamine or protamine alone, each in a volume of 30 μL, topically on the dorsum of one hind paw, at the site of the i.d. injection. Protamine was used at a concentration of 2 μg/μl and indomethacin was used at a concentration of 1 μg/μl. Mechanical nociceptive threshold was evaluated before carrageenan administration, and again 30 and 90 minutes after. Topical indomethacin attenuated carrageenan-induced hyperalgesia. Two-way repeated measures ANOVA, F₍2.14)=40.46, P<0.0001. Bonferroni's multiple comparison post-hoc test. Data shown as mean±SEM; n=3 paws, per group.

All references, patents, and patent applications disclosed herein are incorporated by reference with respect to the subject matter for which each is cited, which in some cases may encompass the entirety of the document.

What is claimed is:

1. A composition comprising (i) a nonsteroidal anti-inflammatory drug (NSAID), an opioid, or a combination thereof, and (ii) one or more transdermal delivery agents.

2. The composition of claim 1, wherein the one or more transdermal delivery agents comprises DMSO, protamine, terpenes, or a combination thereof.

3. The composition of claim 2, wherein the composition comprises 20-80% DMSO by volume.

4. The composition of any one of claims 1-3, wherein the one or more transdermal delivery agents comprises protamine.

5. The composition of any one of claims 1-4, wherein the one or more transdermal delivery agents comprises one or more terpenes.

6. The composition of claim 5, wherein the one or more terpenes comprises one or more ginkolides.

7. The composition of any one of claims 1-6, wherein the ratio of the nonsteroidal anti-inflammatory drug (NSAID), an opioid, or a combination thereof to the one or more transdermal delivery agents is about 0.001 μg per 1 μL to about 10 μg per 1 μl.

8. The composition of any one of claims 1-6, wherein the ratio of the nonsteroidal anti-inflammatory drug (NSAID), an opioid, or a combination thereof to the one or more transdermal delivery agents is, is about, is less than about, or is greater than about 0.001 μg per 1 μl, 0.005 μg per 1 μl, 0.010 μg per 1 μl, 0.05 μg per 1 μl, 0.10 μg per 1 μl, 0.5 μg per 1 μl, 1 μg per 1 μl, 2 μg per 1 μl, 5 μg per 1 μl, or 10 μg per 1 μl.

9. The composition of any one of claims 1-8, wherein the opioid is oxycodone, oxymorphone, hydrocodone, hydromorphone, fentanyl, sufentanil, alfentanil, remifentanil, morphine, codeine, methadone, tramadol, buprenorphine, meperidine, heroin, tapentadol, oliceridine, nalmefene, naloxone, naltrexone, any compound that targets mu-, kappa-or delta-opioid receptors, or a combination thereof.

10. The composition of any one of claims 1-9, wherein the NSAID is actron, aleve, anaprox, ansaid, aspirin acetylsalicylic acid, brufen, butazolidin, cataflam, ceeoxx, celebrex, celecoxib, ceoxx, choline magnesium trisalicylate, clinoril, clotam, daypro, dayrun, dexdetoprofen, diclofenac, diflusinal, disalcid, dolobid, duraprox, dynastat, etodolac, etoricoxib, feldene, fenoprofen, firocoxib, flurbiprofen, flurwood, froben, ibuprofen, indomethacin, keral, ketoflam, ketrolac, licofelone, lodine, lornoxicam, loxoprofen, loxonin, loxomac, lumiracoxib, meclomen, meclofenamic acid, meclofenemate, medipren, mefenamic acid, melox, meloxicam, mesulid, midol, mobic, mobiflex, mono-gesic, motrin, movalis, nabumetone, naprelan, naprosyn, naproxen, nimalox, nimesulide, nuprin, nurofen, orudis, oruvail, oxaporozin, oxeno, parecoxib, phenylbutazone, piroxicam, ponstel, previcox, prexige, rapid, recoxa, relafen, rofecoxib, salflex, salicylate, salsalate, salsitab, sprix, sulide, sulindac, tenoxicam, tolectin, tolfenamic acid, toradol, trilisate disaclid, tufnil, urbifen, valdecoxib, vioxx, voltaren xefo, any compound that targets COX-1 or COX-2, or a combination thereof.

11. A method of treating a pain condition in a subject in need thereof, the method comprising topically administering to the subject the composition of any one of claims 1-10.

12. The method of claim 11, wherein the pain condition is neuropathic pain, inflammatory pain, chemotherapy-induced painful peripheral neuropathy (CIPN), stress-induced pain, or a combination thereof.

13. A composition comprising (i) an antihistamine, a kappa opioid receptor agonist, an anti-pruritogen, or a combination thereof, and (ii) one or more transdermal delivery agents.

14. The composition of claim 13, wherein the one or more transdermal delivery agents comprises DMSO, protamine, terpenes, or a combination thereof.

15. The composition of claim 14, wherein the composition comprises 20-90% DMSO by volume.

16. The composition of any one of claims 13-15, wherein the one or more transdermal delivery agents comprises protamine.

17. The composition of any one of claims 13-16, wherein the one or more transdermal delivery agents comprises one or more terpenes.

18. The composition of claim 17, wherein the one or more terpenes comprises one or more ginkolides.

19. The composition of any one of claims 13-18, wherein the ratio of the antihistamine, the kappa opioid receptor agonist, the anti-pruritogen, or combination thereof to the one or more transdermal delivery agents is about 0.001 μg per 1 μL to about 10 μg per 1 μl.

20. The composition of any one of claims 13-18, wherein the ratio of the antihistamine, the kappa opioid receptor agonist, the anti-pruritogen, or combination thereof to the one or more transdermal delivery agents is, is about, is less than about, or is greater than about 0.001 μg per 1 μl, 0.005 μg per 1 μl, 0.010 μg per 1 μl, 0.05 μg per 1 μl, 0.10 μg per 1 μl, 0.5 μg per 1 μl, 1 μg per 1 μl, 2 μg per 1 μl, 5 μg per 1 μl, or 10 μg per 1 μl.

21. The composition of any one of claims 13-20, wherein the antihistamine is chlorpheniramine, brompheniramine, dexchlorpheniramine, tripolidine, clemastine, diphenhydramine, promethazine, piperazines, piperidines, astemizole, loratadine and terfenadine. Further examples of antihistamines contemplated by the disclosure include, but are not limited to, Brompheniramine (Dimetane), Cetirizine (Zyrtec), Chlorpheniramine (Chlor-Trimeton), Clemastine (Tavist), Diphenhydramine (Benadryl), Fexofenadine (Allegra), or Loratadine (Alavert, Claritin)); adrenaline, or a combination thereof.

22. The composition of any one of claims 13-21, wherein the kappa opioid receptor agonist is JT09, nalfurafine, CR665, or CR845, nalbuphine, butorphanol, pentazocine, asimadoline, enadoline, fedotozine, spiradoline, nalfurafine, noribogaine, niravoline, difelikefalin, apadoline, salvinorin A, levorphanol, levallorphan, phenazocine, eptazocine, CR665 (FE-200665, JNJ-38488502), or a combination thereof.

23. The composition of any one of claims 13-22, wherein the anti-pruritogen is hydroxyzine, benadryl, diphenhydramine, gabapentin, vistaril, hydrocortisone, doxepin, ondansetron, ammonium lactate, calamine, cyproheptadine, diphenhyramine, amlactin, banophen, cortizone, pramoxine, ala-cort, diphedryl, eurax, lac-hydrin, xylocaine, ala-scalp, allermax, anecream, anusol, aquanil, aquax, bactine, cidaleaze, crotamiton, dermacinrx lidotral, dermacinrx lido v pak, dermarest plus anti-itch, dermtex hc, dicopanol, diphen, dytuss, eha lotion, gen7t lotion, gynecort, itch-x lotion, itch relief, lidamantle, lidogel, lidozion, locoid, lydexa, nalbuphine, difelikefalin, asamadoline, nalfurafine, butorphanol, pentazocine, nucort, pandel, proctozone, prudoxin, regencare, sarna sensitive, scot-tussin, texacorttwilite, u-cort, valu-dryl, vanamine, pdzonalon, ala-quin, ana-lex, anacaine, anamantle, benzocaine, chiggerex, geri-hydrolac, gold bond, kerasal al, lanacane, menthol, pramox, prax, radiaura, or a combination thereof.

24. A method of treating pruritus in a subject in need thereof, the method comprising topically administering to the subject the composition of any one of claims 13-23.


SEQUENCE LISTING (11 sequences)

SEQ ID NO: 1 - protein, 11 aa, synthetic construct
YARAAARQARA

SEQ ID NO: 2 - protein, 21 aa, synthetic construct
WLRRIKAWLRRIKAWLRRIKA

SEQ ID NO: 3 - protein, 11 aa, synthetic construct
YGRKKRRQRRR

SEQ ID NO: 4 - protein, 16 aa, synthetic construct
RQIKIYFQNRRMKWKK

SEQ ID NO: 5 - protein, 34 aa, synthetic construct
DAATATRGRSAASRPTERPRAPARSASRPRRPVE

SEQ ID NO: 6 - protein, 16 aa, synthetic construct
AAVALLPAVLLALLAP

SEQ ID NO: 7 - protein, 26 aa, synthetic construct
GWTLNSAGYLLKINLKALAALAKKIL

SEQ ID NO: 8 - protein, 11 aa, synthetic construct
RRRRRRRRRRR

SEQ ID NO: 9 - protein, 18 aa, synthetic construct
KLALKLALKALKAALKLA

SEQ ID NO: 10 - protein, 15 aa, Flock House virus
RRRRNRTRRNRRRVR

SEQ ID NO: 11 - protein, 21 aa, synthetic construct
KETWWETWWTEWSQPKKKRKV